(19) United States
(12) Patent Application Publication
(10) Pub. No.: US 2007/0129615 A1
(43) Pub. Date: Jun. 7, 2007

(54) Apparatus for recognizing abnormal tissue using the detection of early increase in microvascular blood content

(75) Inventors: Vadim Backman, Chicago, IL (US); Hemant Roy, Highland Park, IL (US); Young L. Kim, Skokie, IL (US); Yang Liu, Somerset, NJ (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignees: Northwestern University, Evanston, IL (US); Evanston Northwestern Healthcare, Evanston, IL (US)

(21) Appl. No.: 11/604,659

(22) Filed: Nov. 27, 2006

Related U.S. Application Data
(63) Continuation-in-part of application No. 11/261,452, filed on Oct. 27, 2005.
(60) Provisional application No. 60/801,947, filed on May 19, 2006.

(51) Int. Cl. A61B 5/00

(52) U.S. Cl. 600/315

(57) ABSTRACT

The present invention relates to probe apparatuses and component combinations thereof that are used to recognize possibly abnormal living tissue using a detected early increase in microvascular blood supply and corresponding applications. In one embodiment there is disclosed an apparatus that emits broadband light obtained from a light source onto microvasculature of tissue disposed within a human body and receives interacted light that is obtained from interaction of the broadband light with the microvasculature for transmission to a receiver. Different further embodiments include combinations of optical fibers, polarizers and lenses that assist in the selection of a predetermined depth profile of interacted light. In another embodiment, a kit apparatus is described that has various probe tips and/or light transmission elements that provide for various combinations of predetermined depth profiles of interacted light. In a further embodiment, a method of making a spectral data probe for a depth range detection selectivity for detection of blood within microvasculature of tissue is described.

### PRIORITY CLAIM

This application claims priority to U.S. Application No. 60/801,947 entitled “Guide-To-Colonoscopy By Optical Detection Of Colonic Micro-Circulation And Applications Of Same”, which was filed on May 19, 2006, the contents of which are expressly incorporated by reference herein. This application is also a continuation-in-part and claims priority to co-pending U.S. patent application Ser. No. 11/261,452 entitled “Multi-Dimensional Elastic Light Scattering”, filed Oct. 27, 2005 with the same assignee as the present invention, the disclosure of which is incorporated in its entirety herein by reference.

This application is also related to co-pending U.S. Patent Application with Attorney Docket No. 16936-58277, entitled “Method of Recognizing Abnormal Tissue Using the Detection of Early Increase in Microvascular Blood Content” that is being filed on the same day as this application, and which is also incorporated in its entirety by reference.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### STATEMENT AS TO RIGHTS UNDER FEDERALLY-SPONSORED RESEARCH

This invention was made with Government support under Grant Nos. R01CA109861 and U01CA111257 awarded by the National Institutes of Health of the United States. Accordingly, the United States Government may have certain rights in this invention pursuant to the grant.

### FIELD OF THE INVENTION

The present invention relates generally to light scattering and absorption, and in particular to probe apparatuses and component combinations thereof that are used to recognize possibly abnormal living tissue using a detected early increase in microvascular blood supply and corresponding applications including in vivo tumor imaging, screening, detecting and treatment, and, in particular, “Early Increase in microvascular Blood Supply” (EIBS) that exists in tissues that are close to, but are not themselves, the lesion or tumor and in tissues that precede the development of such lesions or tumors.

### BACKGROUND OF THE INVENTION

Optical probes are known that detect optical signals. Simple optical probes will transmit broadband or a laser light to a target with one optical fiber, and receive the light such as light that is elastically scattered from a specimen, fluorescent light, Raman scattered light, etc., with another optical fiber. The received backscattered light can be channeled to a receiver, such as a CCD array, and the spectrum of the signal is recorded therein.

While such probes work sufficiently for their intended purposes, new observations in terms of the type of measurements that are required for diagnostic purposes have required further enhancements and improvements. The present invention sets forth enhancements and improvements for a variety of different probes that are useful in detecting “Early Increase in microvascular Blood Supply” (EIBS).

### SUMMARY OF THE INVENTION

The present invention relates generally to light scattering and absorption, and in particular to probe apparatuses and component combinations thereof that are used to recognize possibly abnormal living tissue using a detected early increase in microvascular blood supply and corresponding applications

In one embodiment there is disclosed an apparatus that emits broadband light obtained from a light source onto microvasculature of tissue disposed within a human body and receives interacted light that is obtained from interaction of the broadband light with the microvasculature for transmission to a receiver. Different further embodiments include combinations of optical fibers, polarizers and lenses that assist in the selection of a predetermined depth profile of interacted light.

In another embodiment, a kit apparatus is described that has various probe tips and/or light transmission elements, such that each different combination preferably provides for a different predetermined depth profile of interacted light.

In a further embodiment, a method of making a spectral data probe for a depth range detection selectivity for detection of blood within microvasculature of tissue is described.

### BRIEF DESCRIPTION OF THE DRAWINGS

These and other aspects and features of the present invention will become apparent to those of ordinary skill in the art upon review of the following description of specific embodiments of the invention in conjunction with the accompanying figures, wherein:

FIGS. 1(a) and (b) show schematically according to one embodiment of the present invention a fiber-optic polarization-gated probe: (a) side view and (b) cross-section view of distal (i.e., close to tissue surface) tip.

FIG. 2 shows according to one embodiment of the present invention photographically a polarization-gated probe in an accessory channel of an endoscope.

FIG. 3 illustrates a probe kit according to the present invention.

FIGS. 4(a)-(j) illustrate various configurations of the probe according to the present invention.

### DETAILED DESCRIPTION OF THE PREFERRED EMBODIMENTS

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views, As used in the description herein and throughout the claims that follow, the meaning of “a”, “an”, and “the” includes plural reference unless the context clearly dictates otherwise Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention, For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, not is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The present invention, in one aspect, relates to a probe apparatus that is used for optically examining a target for tumors or lesions using what is referred to as “Early Increase in microvascular Blood Supply” (EIBS) that exists in tissues that are close to, but are not themselves, the lesion or tumor. While the abnormal tissue can be a lesion or tumor, the abnormal tissue can also be tissue that precedes formation of a lesion or tumor, such as a precancerous adenoma, aberrant crypt foci, tissues that precede the development of dysplastic lesions that themselves do not yet exhibit dysplastic phenotype, and tissues in the vicinity of these lesions or pre-dysplastic tissues.

A particular application described herein is for detection of such lesions in colonic mucosa in early colorectal cancer (“CRC”), but other applications are described as well.

The target is a sample related to a living subject such as a human being or animal. The sample may be a part of the living subject such that the sample is a biological sample, wherein the biological sample may have tissue developing a cancerous disease.

The neoplastic disease is a process that leads to a tumor or lesion, wherein the tumor or lesion is an abnormal living tissue (either premalignant or cancerous), such as pancreatic cancer, a colon cancer, an adenomatous polyp of the colon, a liver cancer, a lung cancer, a breast cancer, or other cancers.

The measuring step is preferably performed in vivo, though it can be performed ex vivo as well. The measuring step may further comprise the step of acquiring an image of the target. The image, obtained at the time of detection, can be used to later analyze the extent of the tumor, as well as its location.

In one embodiment, the probe projects a beam of light to a target that has tissues with blood circulation therein. At least one spectrum of light scattered from the target is then measured, and blood supply information related to the target is obtained from the measured at least one spectrum. The obtained blood supply information comprises data related to at least one of blood content, blood oxygenation, blood flow and blood volume.

The probe can be used to obtain different optical measurements. According to one embodiment, it may be used to obtain a first set of the blood supply information from a first location of the target and then obtain a second set of the blood supply information from a second location of the target. The first set of the blood supply information at a first location of the target and the second set of the blood supply information at a second location of the target can then be compared to determine the status of the target. One can compare the data to indicate whether the tumor or lesion exists at all by comparison to previously established microvascular blood content values from patients who harbor neoplasia and from those who are neoplasia free.

In one embodiment, a probe apparatus comprises a light source configured and positioned to project a beam of light to a target; and means for measuring at least one spectrum of light scattered from the target; and means for obtaining blood supply information related to the target from the measured at least one spectrum.

The probe apparatus may further comprise a detector that obtains a first set of the blood supply information at a first location of the target. The same detector can be used to obtain a second set of the blood supply information at a second location of the target. Spectral data which is then obtained from the probe apparatus is analyzed and used to determine whether the tissue that has been inspected is abnormal. This analysis is fully described in the application referred to earlier as co-pending U.S. Patent Application with Attorney Docket No. 16936-58277, entitled “Method of Recognizing Abnormal Tissue Using the Detection of Early Increase in Microvascular Blood Content” that is being filed on the same day as this application, and as such is not described further herein.

In one embodiment, at least one spectrum of light scattered from the target is measured by a fiber optic probe according to the present invention, wherein the fiber optic probe comprises a polarization-gated fiber optic probe configured to detect the blood content information. The light source comprises an incoherent light source (such as a xenon lamp).

In one embodiment, the fiber optic probe includes a proximal end portion, an opposite, distal portion, and a body portion with a longitudinal axis defined between the proximal end portion and the distal portion. The body portion is formed with a cavity along the longitudinal axis. At least one first type of fiber is used for delivering a beam of energy to a target, wherein the at least one first type fiber is at least partially positioned within the cavity of the body portion. An optical element is positioned at the proximal end portion and configured to focus the beam of energy to the target. At least one second type fiber is used for collecting scattered energy from the target, wherein the at least one second type fiber is at least partially positioned within the cavity of the body portion.

The fiber optic probe may further comprise at least one linear polarizer optically coupled to the at least one first type fiber and the at least one second type fiber and positioned proximate to the proximal end portion, and wherein the optical element is positioned at the proximal end portion and configured to focus the scattered energy from the target to the at least one linear polarizes for the at least one second type fiber to collect.

The optical element comprises at least one of a ball lens, a graded refractive index lens, an aspheric lens, cylindrical lens, convex-convex lens, and plano-convex lens, although preferably just a single lens is used. Lenses other than these above-mentioned lenses can also be used. It is further noted that different lenses can be used to assist in discriminating measurements and to achieve different tissue penetration depths. Thus, for example, to achieve the shortest penetration depth, a lens can be positioned at the focal distance from the end of the light-collecting fibers with the fibers positioned symmetrically around the axis of the lens. This configuration further increases the intensity of collected light, particularly when a probe is at a distance from tissue, and provides improved stability of the signals collected by the probe in terms of different distances from tissue (if a probe is not in contact with tissue) and pressures exerted by the probe onto tissue (if a probe is in contact with tissue). Shorter penetration depth can also be achieved by using a lens with a shorter focal distance, smaller numerical aperture of the illumination and/or collection fibers, and larger distance between illumination and collection fiber. In principle, penetration depths from a few tens of microns to a few millimeters can be achieved by choosing a proper combination of these probe characteristics.

The at least one first type fiber comprises an illumination fiber, wherein the illumination fiber is optically coupled to the light source.

The at least one second type fiber can also be formed with one or more collection fibers, wherein the one or more collection fibers are optically coupled to an imaging spectrograph and a CCD at the distal end portion, which imaging spectrograph is used to obtain an image of the target. The body portion comprises a tubing.

The following further details of the preferred embodiments that will further describe the invention.

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

Polarization Gating: Polarization gating has been previously used to selectively record short-traveling photons as well as to increase contrast for photons emerging from deeper tissue. As has been shown by our group, the differential polarization signal Δ I (λ)=I∥(λ)−I^{⊥}(λ) is primarily contributed by scatterers located close to the tissue surface and, therefore, particularly sensitive to the properties of the superficial tissues, e. g. epithelial. Our experiments showed that the contribution to the differential polarization signal from deeper tissue structures rapidly decreases with “optical distance” (aka. “optical depth”) to the structure and, hence, with depth (optical distance τ=L/ls with L “physical” depth and ls photon mean free path length in tissue). Because optical density of the epithelium is much smaller than that of underlying connective tissue, in the colon, differential polarization signals are primarily collected from the epithelium plus the mucosal connective tissue with the depth of penetration determined by the design of the probe. For example, if a probe's depth of penetration is about two optical distances or, put alternatively, about two mean free path lengths, it corresponds to approximately the colonic mucosal connective depth of ˜100 μm. This near-surface portion of subepithelial stoma contains a network of capillaries supplying oxygen to the epithelium. Co-polarized signal I∥, arbitrarily polarized signal I∥+I¹⁹⁵ and cross-polarized signal I^{⊥} contain information about progressively deeper tissue, up to several millimeters below the surface for certain probe configurations.

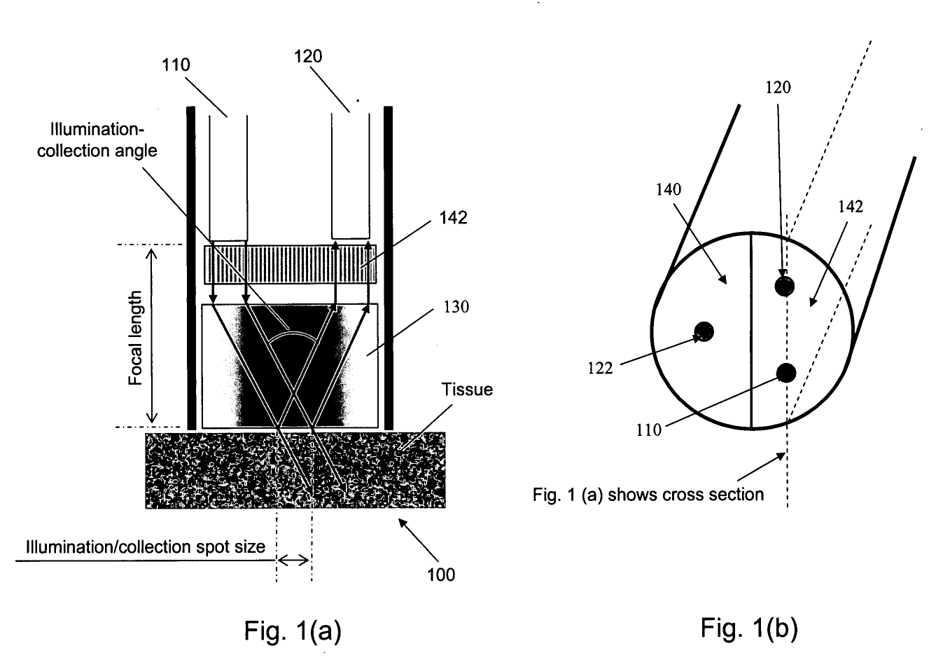

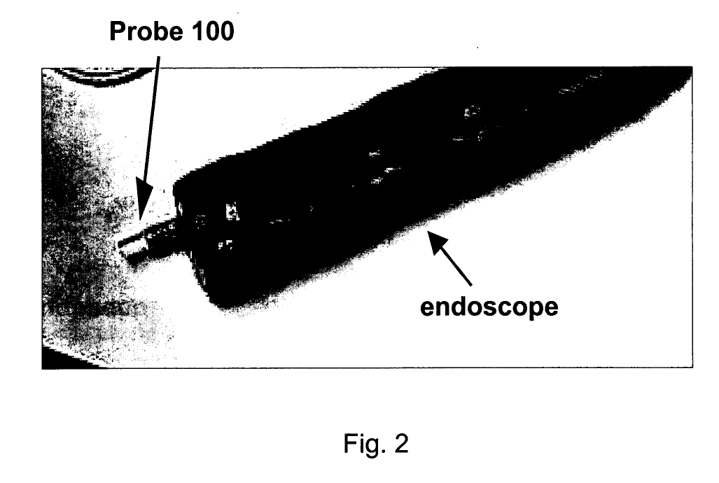

Polarization Gated Fiber-Optic Probe to Detect EIBS: In one aspect, a fiber-optic probe has been developed to accurately detect blood supply in tissue mucosa. FIGS. 1A and 1B illustrate the design of the probe and FIG. 2 shows a photograph of the probe protruding from an accessory channel of a colonoscope. The probe 100 has one or more 100 μm-diameter fibers, one delivery fiber 1 10 used for delivery of linearly polarized light from a Xe-lamp (not shown) onto the tissue surface and the other two fibers 120 and 122 for collecting scattered light from the tissue. A positive lens 130 was positioned at the focal distance from the fiber tips. Several lens types were also tested, including ball, graded refractive index (GRIN), and aspherical lenses. All of the different types of lenses could be used and these provide different performance of the probe in terms of the depth of penetration. In the configuration where the lens 130 was positioned at the focal distance from the fiber tips, it focused light backscattered from a sample onto different fibers 120 and 122, depending on the angle of backscattering. It also ensured that all collection fibers receive scattered light from the same tissue site, which coincides with the illumination spot. The lens 130 does not have to be positioned at the focal distance from the fibers 110, 120, 122, but this configuration provides better performance in terms of 1) shorter penetration depth, in particularly for the polarization gated signal, 2) increases signal level and, thus, time required to collect the signal with sufficient signal-to-noise ratio, 3) prevents collection of specular reflection from probe and tissue surfaces, and 4) improves stability of the measurements in terms of probe displacement from tissue surface in non-contact geometry or the pressure exerted by the probe onto a sample. In the proximal end of the probe 100, the collection fibers 120, 122 are coupled to an imaging spectrograph and a CCD. Two thin film polarizers 140, 142 were mounted on the proximal tip of the probe to polarize the incident light and enable collection of both polarization components (i.e. parallel I∥ and I^{⊥} perpendicular to the incident polarization) of the backscattered light to allow for polarization gating. All components of the probe 100 were made from FDA approved materials.

At least some or all of the components of the probe 100 can be selected according to their characteristics or variables such as optical parameters, relative positions, geometrical dimensions to assist with the detection of the interacted light at different tissue penetration depths. A lens at the probe tip is one of these components that assist in allowing the selecting of a desired penetration depth. For example, to achieve a shorter penetration depth, a lens can be positioned at the focal distance from the end of the fibers with the fibers positioned symmetrically around the axis of the lens. Furthermore, one can use a lens with a shorter focal distance, smaller numerical aperture of the illumination and/or collection fibers, and a larger distance between the illumination and collection fiber. For example, probes were fabricated with a GRIN lens with the penetration depth in colon tissue for polarization-gated signal ˜85 microns (˜1.7 mean free path lengths) and that for cross-polarized light ˜260 microns. A ball lens probe with penetration depths ˜23 and 275 microns was also developed. As such, it is apparent that penetration depths from a few tens of microns to a few millimeters can be achieved by choosing a proper combination of probe characteristics.

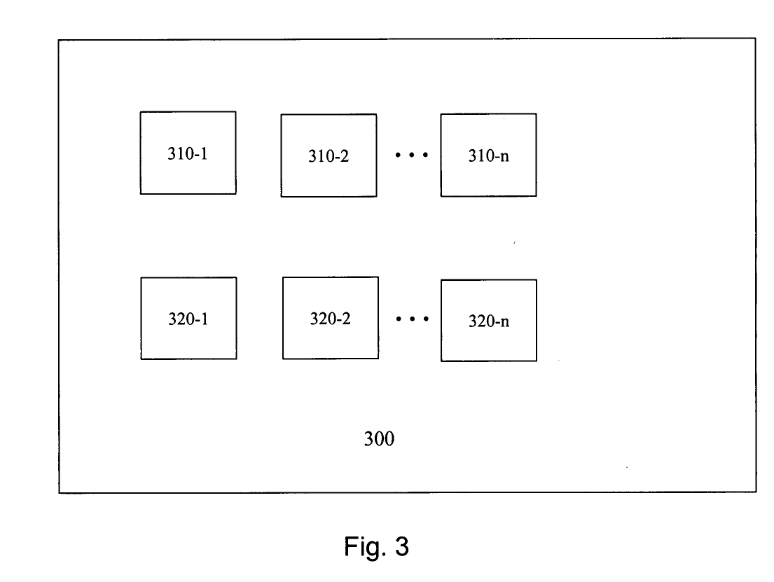

Accordingly, the present invention provides for a probe kit 300, illustrated in FIG. 3 that contains a plurality of interchangeable probe tips 310-1 to 310-n and a plurality of interchangeable optical transmission elements 320-1 to 320-n, where n is an integer greater than 1. Different combinations of these allow for a variety of depth selectivity.

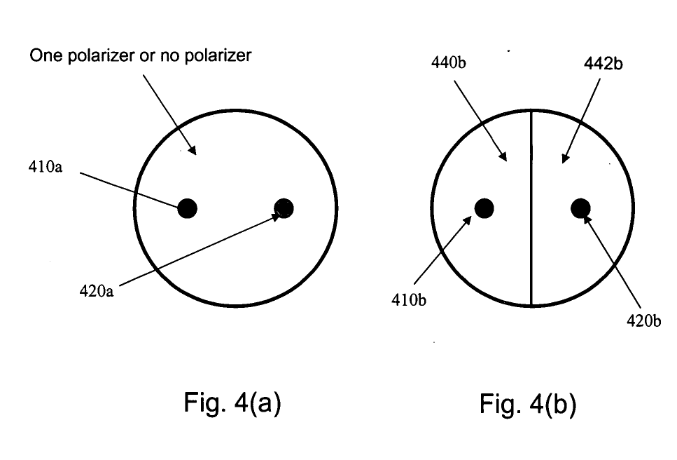

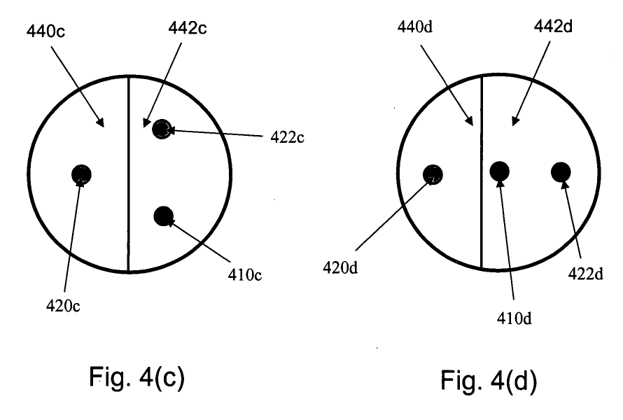

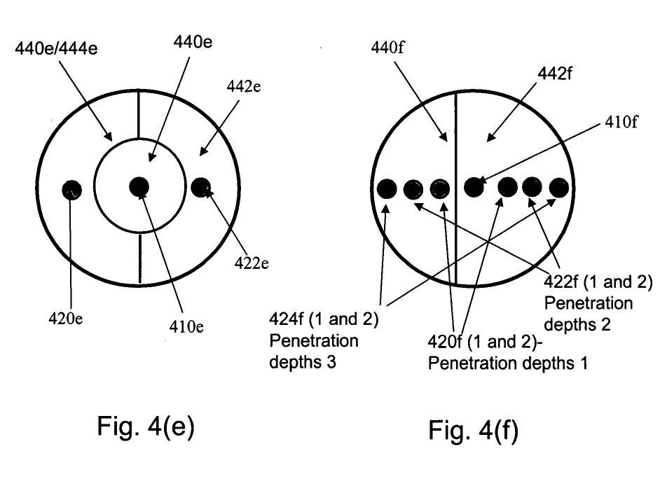

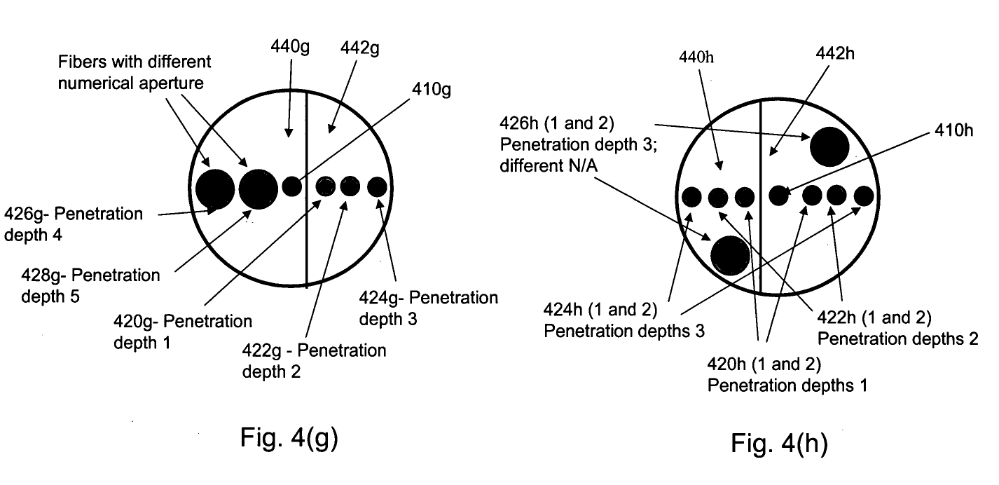

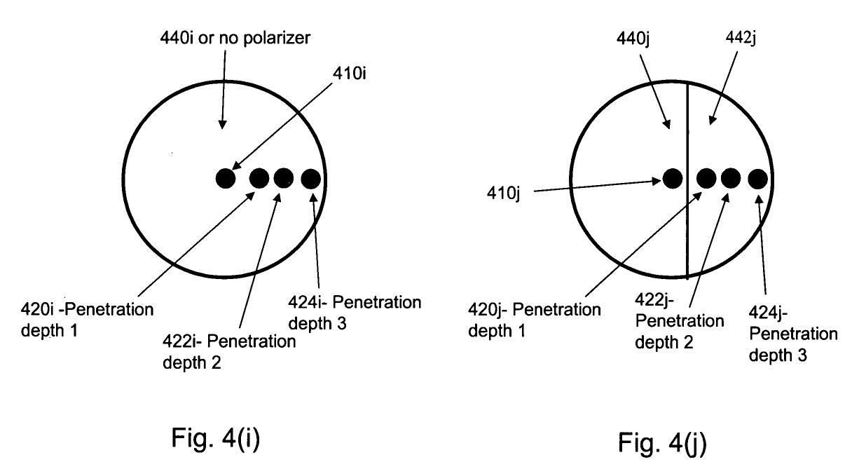

FIGS. 4(a)-(j) illustrate various configurations of the probe according to the present invention. FIGS. 4(a)-(e) illustrate probe configurations that have a single depth selectivity based upon the various characteristics of the components included. FIG. 4a shows an embodiment in which there is only a single delivery fiber 410a and a single collection fiber 420a. There may be a polarizer 440a. FIG. 4b is similar to FIG. 4a, but further includes the usage of two polarizers 440b and 442b. FIGS. 4c, 4d and 4e illustrate versions with two collection fibers 420c, 422c; 420d, 422d; and 420e, 422e, respectively. In each of these embodiments there are two or three polarizers, 440c and 442c; 440d and 442d; and, 440e, 442e and 444e as shown, respectively, in various configurations relative to the optical fibers.

FIGS. 4(f)-(j) illustrate probe configurations in which a single probe, including both the probe tip and the transmission delivery element, can have more than one depth selectivity.

In FIG. 4(f) there exist pairs of collection fibers, and each pair has the same collection depth. Thus collection pair 420f1 and 420f2 have penetration depth 1, collection pair 422f1 and 422f2 have penetration depth 2, and collection pair 424f1 and 424f2 have penetration depth 3. Each of the fibers in each pair is spaced the same distance from the delivery optical fiber 410f. There are two polarizers 440f and 442f as shown.

In FIG. 4(g) there is not a collection pair, but rather individual collection fibers 420g, 422g, 424g, 426g and 428g, that each has a different spacing from the delivery optical fiber 410g. Certain of the collection optical fibers have a different numerical aperture than the others, shown as 426g and 428g. There are two polarizers 440g and 442g as shown.

The FIG. 4(h) embodiment is similar to the FIG. 4(f) embodiment, except at the penetration depth 3 there is an additional fiber pair that includes collection optical fibers 426h1 and and 426h4, each of which has a numerical aperture different that the other collection pair at penetration depth 3. There are two polarizers 440h and 442h as shown.

FIG. 4(i) illustrates a probe having a delivery fiber 410i, and three collection fibers 420i, 422i and 424i, each spaced a different distance from the delivery fiber 410i. There is either no polarizer or one polarizer (not labeled).

FIG. 4(j) illustrates a probe that is same as the probe illustrated in FIG. 4(i) except that it includes two polarizers 440(j) and 442(j), rather than none or one polarizer.

The following discussion sets forth the light path for a three-fiber, two polarizer version of a probe, such as illustrated in FIG. 4(b)-(e). A lamp/light source emits unpolarized light. This light is coupled into a delivery fiber 410. Unpolarized light emerges from this fiber 410 and passes through the first polarizer 420 and becomes linearly polarized. This light is diverging with angle of divergence depending on the numerical aperture (NA) of the fiber 410. Typical NA is about 0.22, which means that the angle of divergence is ˜25 degrees. Fibers with NA's between 0.1 and 0.5 are also available. This polarized but diverging beam then passes through a lens 430, gets collimated, and impinges upon tissue. The lens 430 is positioned at a focal distance from the fibers 410 and 440. Two collection fibers 440 collect the light that interacts with, such as by backscattering, the tissue. The spot on tissue surface, which is formed such that the light that emerges from tissue from this spot can reach and can get collected by one of the collection fibers 440, will be referred to as the “collection spot” for a given collection fiber 440. If tissue surface is in the focal plane of the lens 430 (GRIN lenses typically have the focal planes coinciding with their surfaces) all illumination and collection spots coincide. One of the collection fibers 440 shares the same polarizer 420 with the delivery fiber 410 and the other fiber 440 is “behind” a second polarizer 450 with the axis of polarization orthogonal (or, to be more general, just different) to the axis of polarization of the first polarizer 420. The light that interacts, such as by being backscattered, from tissue has both polarization components. Each of these polarizers 420 and 450, selects light polarized in a particular way and only this light reaches the corresponding collection fiber 440. The first fiber 440 collects light that is polarized along the same direction as the incident light. This is co-polarized light. The other fiber 440 collects the cross-polarized light. On the other (proximal) ends, the light transmitted through the collection fibers 440 is coupled into a spectrometer and a detector (not shown). The spectrometer and a detector can be a single linear array detector (one for each fiber) or an imaging spectrometer and a CCD (which is more expensive). The detector records the spectrum of light intensity from each fiber, which becomes the co-polarized (I_{∥}) and cross-polarized signals/spectra (I_{⊥}). These spectra are then transmitted to a computer or a CPU. The computer can process these spectral data. Four different spectral curves can be looked at: 1) Differential polarization (or what is called polarization-gated) signal is calculated as I_{∥}−I_{⊥}; 2) The total (or arbitrarily polarized) signal is found as I_{∥}+I_{⊥}; 3) Co-polarized signal I₈₁; and 4) Cross-polarized signal I_{⊥}. Each of these four signals is preferentially sensitive to tissue up to its own penetration depth. In principle, one does not have to use two polarizers and measure both co- and cross-polarized signals. If shallow penetration depth is not desired, one can use just a single polarizer and only one collection fiber (co-polarized signal only), two polarizers and collect cross-polarized signal, no polarizer and collect I_{∥+}I_{⊥}, etc.

Specific characteristic combinations for two different probes 100 are provided below.

### EXAMPLE 1

### Ball Lens Probe

Ball lens diameter: 2 mm,
focal length: 1.1 mm,
fiber core diameter: 200 microns,
numerical aperture (NA): 0.22,
distance between illumination and detection fibers: 0.5 mm,
spot size on tissue surface: 0.5 mm,
output (incident on tissue) beam divergence: 5 deg,
outer diameter of the probe: 2.6 mm.

### EXAMPLE 2

### GRIN Lens Probe

GRIN lens diameter: 1.8 mm,
focal length: 2.4 mm,
fiber core diameter: 200 um,
NA: 0.22,
distance between illumination and detection fibers: 0.7 mm,
spot size: 0.7 mm,
output beam divergence: 3 deg,
outer diameter of the probe: 2.5 mm.

In addition to modifying characteristics of the probe tips and the optical transmission elements as mentioned above, there are other characteristics that can be modified.

These modifications include altering where the end of the collection fiber is relative to the delivery fiber in order to get different angular ranges for this depth selective probe. Positioning fibers away from the focal distance is essentially equivalent to using fibers with a larger diameter that are positioned in the focal plane, which in turn is equivalent to a greater divergence of incident or collected light beams. Greater divergence results in addition of both longer and shorter light paths inside tissue. Overall, in most cases this will result in deeper penetration. It should be noted, however, that by “defocusing” the probe, the “defocusing” configuration is less efficient in terms of intensity the probe collects.

Another characteristic is the distance to the tissue surface. This distance can be controlled by choosing a proper spacer between the lens and the tissue surface. (As discussed above, most GRIN lenses have their focal plane coinciding with their sides but this is not necessarily the case with other lens types. If other lenses are used, one can put a spacer to distance the probe from tissue.) If this distance is different from the focal length, penetration depth is greater.

The foregoing description of the exemplary embodiments of the invention has been presented only for the purposes of illustration and description and is not intended to be exhaustive or to limit the invention to the precise forms disclosed. Many modifications and variations are possible in light of the above teachings.

What is claimed

1. An apparatus that emits broadband light obtained from a light source onto microvasculature of tissue of a human body and receives interacted light that is obtained from interaction of the broadband light with the microvasculature for transmission to a receiver, the apparatus comprising:
a probe having an end adapted for insertion into the human body and which illuminates the tissue with the broadband light and receives interacted light that interacts with blood in the microvasculature that is within the tissue, the probe including:
a delivery optical fiber having a delivery numerical aperture for transmitting the broadband light obtained from the light source, the delivery optical fiber having a light delivery end adapted for emission of the broadband light and a light delivery source connection end adapted for connection to the light source;
at least one collection optical fiber having a collection numerical aperture, the collection optical fiber having a light collection end that receives the interacted light and a receiver connection end adapted for connection to the receiver, wherein the light collection end is substantially aligned with and at a predetermined distance from the light delivery end of the delivery optical fiber; and
a lens that is spaced substantially about one focal length of the lens from the light collection end of the collection optical fiber and from the light delivery end of the delivery optical fiber;
wherein the delivery optical fiber and the collection optical fiber and the lens are adapted to collect the interacted light at a collection spot on a surface of the tissue that is within the focal plane of the lens, and wherein the interacted light collected results from interactions with the microvasculature that are substantially at a predetermined penetration depth below the collection spot, and wherein the predetermined penetration depth is obtained in part due to a selected plurality of characteristics, the selected plurality of characteristics including selection of the focal length of the lens and at least one further characteristic from one of the delivery optical fiber, the collection optical fiber, and the lens.

2. The apparatus according to claim 1 wherein the at least one further characteristic is a characteristic of one of the delivery optical fiber and the collection optical fiber, and is either (1) a type of the delivery optical fiber, (2) a type of the collection optical fiber, (3) the delivery and collection numerical apertures, (4) the substantial alignment of the light delivery end of the delivery optical fiber and the light collection end of the collection optical fiber, and (4) the predetermined distance between the light delivery end of the delivery optical fiber and the light collection end of the collection optical fiber.

3. The apparatus according to claim 1 wherein the at least one further characteristic is a lens characteristic and is at least one of (1) a type of lens and (2) a spacing between the lens and the tissue.

4. The apparatus according to claim 1 wherein there is one and only one collection optical fiber.

5. The apparatus according to claim 4 wherein the delivery numerical aperture and the collection numerical aperture are the same.

6. The apparatus according to claim 1 wherein there are two and only two collection optical fibers.

7. The apparatus according to claim 6 wherein the two collection optical fibers have the same collection numerical aperture and the light collection end of each collection optical fiber is substantially aligned with and substantially at a same predetermined distance from the light delivery end of the delivery optical fiber.

8. The apparatus according to claim 7 further including first and second polarizers disposed between the lens and the delivery and collection optical fibers, the first polarizer providing polarization of the emitted broadband light and the interacted light that is directed to one of the two collection optical fibers and the second polarizer providing polarization of the interacted light that is directed to the other of the two collection optical fibers, and wherein the inclusion of the first and second polarizers and the polarization of each are further ones of the selected plurality of characteristics.

9. The apparatus according to claim 7 further including first and second polarizers disposed between the lens and the delivery and collection optical fibers, the first polarizer providing polarization of the emitted broadband light and the second polarizer providing polarization of the interacted light that is directed to at least one of the two collection optical fibers, and wherein the inclusion of the first and second polarizers and the polarization of each are further ones of the selected plurality of characteristics.

10. The apparatus according to claim 9 further including a third polarizer disposed between the other of the two collection optical fibers and the lens, the third polarizer providing polarization of the interacted light that is directed to the other of the two collection optical fibers, and wherein the inclusion of the third polarizer and the polarization of the third polarizer are further ones of the selected plurality of characteristics.

11. The apparatus according to claim 8 wherein the substantial alignment is on a same plane.

12. The apparatus according to claim 11 wherein the substantially about one focal length is one focal length for each of the light collection ends of the collection optical fibers and the light delivery end of the delivery optical fiber.

13. The apparatus according to claim 11 wherein the substantially about one focal length is greater than one focal length for each of the light collection ends of the collection optical fibers and the light delivery end of the delivery optical fiber.

14. The apparatus according to claim 11 wherein the substantially about one focal length is less than one focal length for each of the light collection ends of the collection optical fibers and the light delivery end of the delivery optical fiber.

15. The apparatus according to claim 8 wherein the substantial alignment provides for the delivery end of the delivery optical fiber to protrude farther than the light collection ends of the collection optical fibers.

16. The apparatus according to claim 8 wherein the substantial alignment provides for the light collection ends of the collection optical fibers to protrude farther than the delivery end of the delivery optical fiber.

17. The apparatus according to claim 8 wherein a depth of the blood content in microvascular tissue that is detected is between 0 and 250 rnicrons.

18. The apparatus according to claim 8 wherein the first and second polarizers are orthogonal to each other.

19. The apparatus according to claim 8 wherein the first and second polarizers are at angles to each other that are different than 90 degrees and 45 degrees.

20. The apparatus according to claim 8, the apparatus wherein the receiver collects differently polarized spectral components of the interacted light.

21. The apparatus according to claim 20 wherein the receiver includes means for using the different polarization components to create polarization gated spectral data.

22. The apparatus according to claim 20 wherein the receiver includes a linear array CCD detector.

23. The apparatus according to claim 8 wherein the collection end of each collection optical fiber is symmetrically spaced around the light delivery end of the delivery optical fiber.

24. The apparatus according to claim 8 wherein the light source emits at least two wavelength ranges of light.

25. The apparatus according to claim 24 wherein the two wavelength ranges are such that one of them includes 542, 555 and 576 nm wavelengths and the other includes wavelengths longer than 576 nm.

26. The apparatus according to claim 1 wherein the light source emits at least two wavelength ranges of light.

27. The apparatus according to claim 26 wherein the two wavelength ranges are such that one of them includes 542, 555 and 576 nm wavelengths and the other includes wavelengths shorter than 542 nm.

28. The apparatus according to claim 1 wherein the light source obtains the broadband light from a plurality of narrowband light sources.

29. The apparatus according to claim 1 wherein the probe further includes a polarizer disposed between the lens and the delivery and collection optical fibers, the polarizer allowing for at least one of adjustment of polarization of the emitted broadband light and for polarization of the interacted light.

30. An apparatus according to claim 8, wherein the delivery optical fiber and the collection optical fiber are formed as one interchangeable optical transmission element having a device connection end and a detection end, the detection end including the light delivery end and the light connection end of the delivery optical fiber and the collection optical fiber, respectively;
wherein the lens is formed as one interchangeable probe tip assembly, and wherein there is at least one of (a) a plurality of interchangeable probe tip assemblies including the one interchangeable probe tip assembly and (b) a plurality of interchangeable optical transmission elements including the one interchangeable optical transmission element,
wherein each of the plurality of interchangeable probe tip assemblies and each of the plurality of interchangeable optical transmission elements having a different characteristic selected to assist with the detection of the interacted light at different tissue penetration depths so that coupling of a particular interchangeable fiber transmission element to a particular interchangeable probe tip assembly will provide for detection of interacted light at a particular tissue penetration depth.

31. The apparatus according to claim 30 wherein there is a plurality of probe tip assemblies.

32. The apparatus according to claim 31 wherein at least some of the interchangeable probe tip assemblies further include a polarizer disposed between the lens and the delivery and collection optical fibers, the polarizer providing for a further different characteristic of the at least some probe tip assemblies.

33. The apparatus according to claim 32 wherein the polarizer is adapted for at least one of adjustment of polarization of the emitted broadband light and for polarization of the interacted light.

34. The apparatus according to claim 31 where a further different characteristic is a distance between the device connection end and a focal plane of the lens for at least some of the plurality of probe tip assemblies.

35. The apparatus according to claim 31 where a further different characteristic is a distance between a focal plane of the lens and a surface of the tissue surface.

36. The apparatus according to claim 34 wherein an outer spacer is used to ensure the distance between the focal plane of the lens and the surface of the tissue surface for at least some of the plurality of probe tip assemblies.

37. The apparatus according to claim 31 wherein one lens in one of the probe tip assemblies has a larger focal length than another lens in another of the probe tip assemblies to obtain a larger spot size of the emitted broadband light.

38. The apparatus according to claim 31 wherein at least one of the optical transmission elements includes two and only two collection optical fibers.

39. The apparatus according to claim 31 where a further different characteristic is a distance between light collection end of each of the two collection optical fibers and the light delivery end of the delivery optical fiber.

40. The apparatus according to claim 39 wherein the distance between the light collection end of each of the two collection optical fibers and the light delivery end of the delivery optical fiber is the same.

41. The apparatus according to claim 38 wherein at least some of the interchangeable probe tip assemblies further include first and second polarizers disposed between the lens and the delivery and collection optical fibers, wherein the first and second polarizers providing for a further different characteristic of the at least some probe tip assemblies.

42. The apparatus according to claim 41, wherein the first polarizer provides polarization of the emitted broadband light and the interacted light that is directed to one of the two collection optical fibers and the second polarizer provides polarization of the interacted light that is directed to the other of the two collection optical fibers.

43. The apparatus according to claim 38 where a further different characteristic is a distance between the light connection end and a focal plane of the lens for at least some of the plurality of probe tip assemblies.

44. The apparatus according to claim 38 where a further different characteristic is a distance between a focal plane of the lens and a surface of the tissue surface.

45. The apparatus according to claim 43 wherein an outer spacer is used to ensure the distance between the focal plane of the lens and the surface of the tissue surface for at least some of the plurality of probe tip assemblies.

46. The apparatus according to claim 38 wherein one lens in one of the probe tip assemblies has a larger focal length than another lens in another of the probe tip assemblies to obtain a larger spot size of the emitted broadband light.

47. The apparatus according to claim 38 where a further different characteristic is a distance between the device connection end and a focal plane of the lens for at least some of the plurality of probe tip assemblies.

48. The apparatus according to claim 38 where a further different characteristic is a distance between a focal plane of the lens and a surface of the tissue surface.

49. The apparatus according to claim 48 wherein an outer spacer is used to ensure the distance between the focal plane of the lens and the surface of the tissue surface for at least some of the plurality of probe tip assemblies.

50. The apparatus according to claim 30 wherein there are a plurality of fiber transmission elements.

51. The apparatus according to claim 50 wherein the different characteristic of at least some of the fiber transmission elements is numerical aperture.

52. The apparatus according to claim 5 1wherein each of the delivery and collection optical fibers of a particular fiber transmission element have the same numerical aperture.

53. The apparatus according to claim 50 wherein the different characteristic of at least some of the fiber transmission elements is spacing between the delivery optical fiber and each of the at least two collection fibers.

54. The apparatus according to claim 53 wherein the spacing allows for an angular range detection difference of at least 4 degrees between at least two different ones of the fiber delivery elements.

55. The apparatus according to claim 50 wherein the different characteristic of at least some of the fiber transmission elements is a diameter of each of the delivery and collection optical fibers.

56. The apparatus according to claim 30 wherein there is a plurality of probe tip assemblies and a plurality of fiber transmission elements.

57. The apparatus according to claim 1 wherein the at least one collection optical fiber is a plurality of collection optical fibers, the plurality of optical fibers including a plurality of paired collection optical fibers, each paired collection optical fiber having a distance between the light collection end of each of the two paired collection optical fibers and the light delivery end of the delivery optical fiber that is the same, and each of the plurality of paired collection optical fibers having a different distance.

58. A method of making a spectral data probe for a depth range detection selectivity for detection of blood within microvasculature of tissue, the spectral data probe receiving broadband light from a light source and providing interacted light to a receiver, the method comprising the steps of:
determining the depth range that is desired for the detection of the interacted light; providing the spectral data probe, the spectral data probe including:
a delivery optical fiber for transmitting the broadband light obtained from the light source, the delivery optical fiber having a delivery light output end adapted for connection to the light source and a delivery light source connection end;
a collection fiber group, the collection fiber group including at least one collection optical fiber, each collection optical fiber having a light collection end that receives the interacted light that interacts with the blood in the microvasculature that is within the tissue and a receiver connection end, wherein each light collection end is substantially aligned with and substantially at a predetermined distance from the light delivery end of the delivery optical fiber; and
a lens that is spaced substantially about one focal length of the lens from each light collection end of each collection optical fiber and from the delivery light output end of the delivery optical fiber;
wherein the step of providing selects different characteristics for the delivery optical fiber, the collection fiber group and the lens to assist with allowing the spectral data probe to have the determined depth range.
